(19) United States
(12) Patent Application Publication
(10) Pub. No.: US 2007/0232898 A1
(43) Pub. Date: Oct. 4, 2007

(54) Telescoping Catheter With Electromagnetic Coils for Imaging and Navigation During Cardiac Procedures

(75) Inventors: Rany Huynh, Charlestown, MA (US); Nasser Rafiee, Andover, MA (US); Nareak Douk, Lowell, MA (US); Morgan House, Newfields, NH (US); Alex Hill, Minneapolis, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/693,854

(22) Filed: Mar. 30, 2007

Related U.S. Application Data
(60) Provisional application No. 60/744,033, filed on Mar. 31, 2006.

(51) Int. Cl. A61B 5/05; A61M 25/00; A61B 5/103

(52) U.S. Cl. 600/424; 600/435; 600/587

(57) ABSTRACT

An image guided navigation system for mapping at least one of the shape, size and location of a structure within a heart of a patient, including a catheter system having a first catheter with a longitudinally extending lumen, a second catheter having a longitudinally extending lumen and positioned at least partially within and moveable relative to the lumen of the first catheter, an elongated member positioned at least partially within and moveable relative to a lumen of the second catheter, a first detectable marker located at a generally distal end of the first catheter, a second detectable marker located at a generally distal end of the second catheter, and a third detectable marker located at a generally distal end of the elongated member, wherein at least one of the three detectable markers is axially moveable relative to at least one of the other detectable markers.

### CROSS-REFERENCE TO RELATED APPLICATION

The present application claims priority to U.S. Provisional Application No. 60/744,033, filed Mar. 31, 2006 and titled “Telescoping Catheter With Electromagnetic Coils for Imaging and Navigation During Cardiac Procedures”, the entire contents of which is incorporated herein by reference in its entirety.

### TECHNICAL FIELD

This invention relates generally to medical devices and particularly to a device, system, and method for aiding implantation of a heart valve repair device.

### BACKGROUND

Heart valves, such as the mitral and tricuspid valves, consist of leaflets attached to a fibrous ring or annulus. These valves are sometimes damaged by diseases or by aging, which can cause problems with the proper functioning of the valve. Referring particularly to the mitral valve, the two native mitral valve leaflets of a healthy heart coapt during contraction of the left ventricle, or systole, and prevent blood from flowing back into the left atrium. However, the mitral valve annulus may become distended for a variety of reasons, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in hypertrophy and dilatation of the left ventricle, further enlarging and distorting the shape of the mitral valve. If left untreated, the condition may result in cardiac insufficiency, ventricular failure, and possibly even death.

A common procedure for repairing the mitral valve involves implanting an annuloplasty ring on the atrial surface of the mitral valve annulus. During implantation, the annuloplasty ring is aligned with the valve annulus and then fixedly attached to the valve annulus, typically using a suturing process. The annuloplasty ring generally has a smaller internal area than the distended valve annulus so that when it is attached to the annulus, the annuloplasty ring draws the annulus into a smaller configuration. In this way, the mitral valve leaflets are brought closer together, which provides improved valve closure during systole.

Implanting an annuloplasty ring on a valve annulus can be accomplished using a variety of repair procedures, such as procedures that require indirect visualization techniques to determine the exact location of the heart valve and annuloplasty ring during placement of the ring at the valve annulus. Indirect visualization techniques, as described herein, are techniques that can be used for viewing an indirect image of body tissues and/or devices within a patient. One example of such a technique is referred to as endoscopic visualization, which involves displaying images from endoscopic light guides and cameras within the thoracic cavity on a video monitor that is viewed by a surgeon. Effective use of this method depends on having sufficient open space within the working area of the patient's body to allow the surgeon to recognize the anatomical location and identity of the structures viewed on the video display, which can be difficult to accomplish in certain areas of the heart.

Another indirect visualization technique involves the use of fluoroscopy, which is an imaging technique commonly used by physicians to obtain real-time images of the internal structures of a patient through the use of a fluoroscope. However, some tissues, such as the cardiac tissues, do not readily appear under fluoroscopy, making it very difficult to accurately align the annuloplasty ring prior to its implantation. To improve the visualization of the area of interest, radiopaque contrast dye can be used with x-ray imaging equipment. However, when treating the mitral valve, for example, repeated injections of contrast dye are not practical because of rapid wash-out of the dye in this area of high fluid flow. Additionally, to make high-volume contrast injections of this kind, an annuloplasty catheter system would require multiple lumens, undesirably large lumens, and/or an additional catheter, none of which is desirable during catheterization procedures. Furthermore, multiple high-volume contrast injections are not desirable for the patient due to potential complications in the renal system, where the radiopaque contrast medium is filtered from the blood.

A wide variety of other techniques are available for viewing images of cardiac structures, including ultrasonography such as trans-thoracic echocardiography (TTE), trans-esophageal echocardiography (TEE), cardiac magnetic resonance (CMR) including magnetic resonance imaging (MRI) or magnetic resonance angiography (MRA), and computed tomography (CT) including computer tomography angiography (CTA). However, none of the above techniques, used alone or in combination with other available techniques, provides adequate visualization and guidance during catheter-based valve repair procedures.

Annuloplasty procedures can be further complicated by the structure of the valve annulus and the fact that the annulus can undergo significant movement during procedures performed on a beating heart. Since annuloplasty is performed on a beating heart, care must be taken during both systole and diastole when positioning an annuloplasty ring for fixation. With particular reference again to the mitral valve, the mitral valve leaflets are basically flaps or appurtenances attached to the cardiac muscle tissue, creating a pseudo-annulus. In particular, when the mitral valve is closed during systole, a relatively flat floor of the left atrium is formed; however, during diastole, the mitral valve leaflets open towards the ventricular walls such that, in many cases, the valve annulus is not well defined. That is, the mitral valve annulus lacks a definable shelf or ledge for conveniently locating an annuloplasty ring. Without the direct optical visualization that is provided during surgery, it can be difficult to position an annuloplasty ring in abutment with the superior surface of this poorly defined valve annulus. As a result, an annuloplasty ring may be inadvertently affixed in a misaligned position below, above or angled across the valve annulus when using the non-optical imaging techniques of a catheter-based procedure. Affixing the annuloplasty ring in such a misaligned position could have negative consequences for the patient, such as increasing mitral regurgitation and/or triggering ectopic heart beats.

One possible method for mapping the mitral valve annulus and obtaining real time imaging during beating heart surgery is through the use of electromagnetic (EM) imaging and navigation. With EM navigation, a patient is generally placed on a table having a plurality of sensors either on the surface of the table or at positions around the table. The sensors are connected to a processor and the processor knows the positions of the sensors relative to the table. A patient is then placed on the table and immobilized either by anesthesia, restraints, or both. An elongated flexible device having at least three EM coils spaced along its distal portion can then be inserted into the patient's body (into the vascular system for example). The coils are typically made from extremely small diameter material that can be wound around the outside of the device or wound around an interior layer of the device and then covered with an additional layer of material. A very thin wire (or some other electrically conductive material) can be used to communicate from an external AC power source to each of these coils. Alternatively, wireless sensors can be used, which can eliminate the need to provide a wire to communicate with the EM coils.

As the elongated device is moved through the body, the sensors can detect the EM signal that is created by the moving coil. The processor then calculates the position of the coils relative to each sensor. The location of the sensors can be viewed on a display device, and the EM navigation can be combined with other navigation/visualization technologies so that the location of the EM coils in a patient's body can be viewed in real time. Additional sensors may also be incorporated into a system using EM navigation to improve the accuracy of the system, such as temporarily attaching sensors to a patient's body and/or covering at least a portion of a patient with a blanket that contains additional sensors. The relationship between all of the sensors can be used to produce the image of the patient's body on the table. Examples of methods and systems for performing medical procedures using EM navigation and visualization systems for at least part of an overall navigation and visualization system can be found, for example, in U.S. Pat. No. 5,782,765 (Jonkman); U.S. Pat. No. 6,235,038 (Hunter et al.); U.S. Pat. No. 6,546,271 (Resifeld); U.S. Patent Application No. 2001/0011175 (Hunter et al.); U.S. Patent Application No. 2004/0097805, (Verard et al.), and U.S. Patent Application No. 2004/0097806 (Hunter et al.), the entire contents of which are incorporated herein by reference.

Another method for mapping the mitral valve annulus and obtaining real time imaging during beating heart surgery is through the use of electro-potential navigation. Electro-potential (EP) navigation is similar to EM navigation in that there are multiple sensors on or around a surface on which a patient is positioned, and the sensors are in communication with a processing device. When using EP navigation, however, a low frequency electrical field is created around the patient, and the coils on the instrument are connected to a DC energy source such that there is a constant energy signal emitting from the coils. The coils create a disturbance in the electrical field as they move through the field, and location of the instrument in the 3D coordinate space is calculated by determining the location of the disturbance in the energy field relative to the sensors.

While the methods, systems, and devices described above provide for real time imaging of devices during certain types of medical procedures, they do not provide a device that can be used to deliver other devices for treating cardiac valve disease. Therefore, it would be desirable to provide a device, system, and method that can provide accurate, real time images of devices that are used for during the catheter based implantation of an annuloplasty ring.

### SUMMARY

In one aspect of this invention, a catheter system is provided that has indirect visualizable properties for aiding in the placement of therapeutic devices relative to a heart valve annulus. Such a catheter system is designed to be delivered to particular areas of the heart via the vascular system of a patient. In one aspect of the invention, devices are provided that can be used in the heart during a therapeutic procedure that can be visualized in real time and be used to determine the shape and location of structure within the heart. An example of such a procedure would be repair of a cardiac valve in which the size, shape and location of the valve annulus must be determined.

One particular application of the catheter systems of the invention is for catheter-based implantation of a heart repair or therapeutic device to treat mitral regurgitation. During such a procedure, these catheter systems can be used to determine the size and shape of the mitral valve annulus and to determine that any therapeutic devices used for treating mitral regurgitation are implanted in the correct location. Thus, the catheter systems can be used with treatment and/or repair methods that determine the location of the posterior commissure of a mitral valve, for example.

Another aspect of the present invention is a telescoping, articulating catheter system for use during implantation of devices for treating heart valves. One exemplary catheter system comprises at least a first articulating catheter and a second articulating catheter disposed in a lumen of the first catheter. Each of the articulating catheters includes an EM coil at or near its most distal tip for EM imaging of the system while it is in a patient's body. The system can include a guide wire disposed in the lumen of the second catheter, and the guide wire can also include an EM coil at or near its most distal tip. The guide wire may have a solid construction or may have a longitudinally extending lumen (e.g., the guide wire may be configured as a catheter). The EM coils of such a system can be connected to an external power source, and the catheter system can be connected to a processor that is part of a larger EM navigation system. The EM navigation system can comprise at least a plurality of sensors and/or transmitters having a known location relative to a patient, a processor that can be used to determine the location of the EM coils relative to the sensors, a power source, and a display device for viewing the movement, shape, and location of the catheter system in real time.

One particular exemplary application of the devices, systems, and methods of the invention is a catheter system that can be delivered through the vascular system of a patient and to the left atrium, where it can be used to determine the shape and orientation of a mitral valve annulus. The catheter system can also be used to deliver devices into the left atrium for implantation in the area of a mitral valve annulus. Examples of such devices can be found in the following references: U.S. Patent Application No. 2007/005,1377(Douk et al.); and U.S. Patent Application No. 2007/002,7533 (Douk); the contents of which are incorporated herein by reference.

One particular embodiment of the invention includes a flexible, adjustable, articulating, system for delivering a device to a mitral valve annulus. The delivery system comprises a telescoping system comprising at least two coaxially positioned catheters and a guide wire. Each of the catheters and the guidewire has an electromagnetic coil at or near its distal end. This allows the telescoping system to be tracked in the 3D coordinate space as if it were a single catheter. The system can be used to map the size and shape of a cardiac valve annulus, and this information can be used for placement of a treatment device, for example.

### BRIEF DESCRIPTION OF THE DRAWINGS

The present invention will be further explained with reference to the appended Figures, wherein like structure is referred to by like numerals throughout the several views, and wherein:

FIG. 1 is a cutaway front view of a portion of a heart, illustrating a catheter system of the present invention being delivered to a left atrium;

FIGS. 2 and 2A are perspective views of the distal end of devices that can be used to puncture the septum according to the invention;

FIG. 3 is a partial cutaway view of a portion of the heart, showing an exemplary placement of a catheter system of the invention on a mitral valve annulus;

FIG. 4 is a perspective view of an end portion of a catheter system of the invention;

FIG. 4A is an exemplary image of the catheter system of FIG. 4 as it can appear on a display monitor of an electromagnetic navigation system;

FIG. 5 is a perspective view of an end portion of another catheter system of the invention;

FIG. 5A is an exemplary image of the catheter system of FIG. 5 as it can appear on a display monitor of an electromagnetic navigation system;

FIG. 6 is a perspective view of an end portion of another catheter system of the invention that is capable of tracking movement of an electromagnetic coil;

FIG. 6A is an exemplary image of the catheter system of FIG. 6 as it can appear on a display monitor of an electromagnetic navigation system; and

FIG. 7 is a block diagram illustrating an electromagnetic navigation system according to the invention.

### DETAILED DESCRIPTION

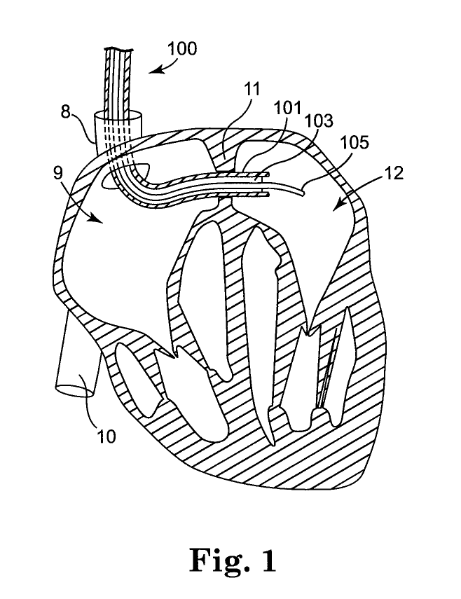

Referring now to the Figures, wherein the components are labeled with like numerals throughout the several Figures, and initially to FIG. 1, one preferred configuration of a catheter system 100 is illustrated as it is being inserted into a heart via the vascular system of a patient. Catheter system 100 generally includes a first catheter 101 having a centrally extending lumen along its length and a second catheter 103 having a centrally extending lumen along its length. A guidewire 105 extends through the length of the lumen of the second catheter 103 and is extendible from the catheter 103 at its distal tip. Although the element 105 is referred to as a guidewire, element 105 can be an elongated member that is a solid wire or can be an elongated member having a longitudinally extending lumen, such as a catheter. If member 105 is a catheter, another element may be positioned within its longitudinally extending lumen, if desired. Each of the catheters 101, 103 and the guidewire 105 have an electromagnetic coil disposed at or near their distal ends, and these electromagnetic coils can be used to help map the shape of mitral valve annulus. Thus, the catheter system 100 includes three electromagnetic coils.

The terms “distal” and “proximal” are used herein with reference to the treating clinician during the use of the catheter system, where “distal” indicates an apparatus portion distant from, or in a direction away from the clinician (e.g., the EM coils will be on the “distal” end of the various system members) and “proximal” indicates an apparatus portion near to, or in a direction towards the clinician. The reference devices of the current invention may be made, in whole or in part, from one or more materials that are viewable by radiography, ultrasound, or magnetic resonance imaging visualization techniques. Embodiments of the devices may also be coated with materials that are visible using such visualization methods.

Much of the description herein relates to use of catheter systems and methods of the invention for placement of device in the heart during mitral valve repair procedures. However, those with skill in the art will recognize that catheter systems of the invention may also be deployed at other cardiac valves or other locations in the body and may be used to visualize/image other structures within the body.

To deliver the catheter system 100 to the desired location in the heart shown in FIG. 1, one exemplary delivery method includes inserting the catheter system 100 into the subclavian vein, through the superior vena cava 8, and into the right atrium 9. Alternatively, the catheter system 100 may be inserted through the femoral vein into the common iliac vein, through inferior vena cava 10, and into right atrium 9. Another possible delivery path would be through the femoral artery into the aorta, through the aortic valve into the left ventricle, and then through the mitral valve into left atrium 12. The delivery path chosen can depend on preferences of the surgeon, constraints provided by the patient's anatomy, and/or other factors.

If the catheter system 100 is being delivered through the superior vena cava 8 and into the right atrium 9, the delivery process includes puncturing the interatrial wall 11 between right atrium 9 and left atrium 12 with a puncturing device, then advancing the distal tip of the catheter system 100 through the septal perforation and into left atrium 12. Depending on the desires of the clinician performing the procedure and the imaging technique/system (or combination thereof) used during navigation to the right atrium 9, the second catheter 103 and guide wire 105 can be extended or they can be withdrawn from an extended position. In one embodiment of the current invention, navigation to the heart is conducted using EM techniques and the system is extended to a sufficient distance that the three EM coils allow the clinician to determine the location of the catheter system in the 3D coordinate space.

The catheter system 100 and other catheter systems of the invention can comprise two or more articulating catheters coaxially oriented such that at least one catheter is inserted into a central lumen of another catheter in a telescopic relationship. That is, the various catheters are moveable relative to each other such that the inner catheter(s) are moveable with respect to the outer catheter(s). In that regard, while the description of catheter systems of the invention are primarily directed to systems having two catheters coaxially positioned relative to each other and a guidewire, it is understood that more than two catheters and/or guidewires can be included in a particular catheter system of the invention.

The catheter system components of the invention are relatively flexible, and configured so that they can be inserted into the cardiovascular system of a patient. They can thus be made of flexible biocompatible materials such as polyurethane, polyethylene, nylon and polytetrafluoroethylene (PTFE). In one embodiment of the invention, the interior surface of catheters of the system are coated with a lubricious material such as silicone, polytetrafluoroethylene (PTFE), or a hydrophilic coating. Other embodiments may include such coatings on the outside surfaces as well. The lubricious surfaces facilitate the longitudinal movement of catheters, guidewires, and therapeutic devices relative to each other and through the system of the patient. Additionally, the catheters of the system can be manipulated/articulated to form a plurality of curves at the distal end thereof, as will be described in further detail below. One advantage of having at least two articulating catheters is that a clinician will be able to mimic and map almost any shape in the body by manipulating the pair of catheters. Another advantage is that the combination of two articulating catheters allows a clinician to aim the distal tip of a second or inner catheter in almost any desired direction.

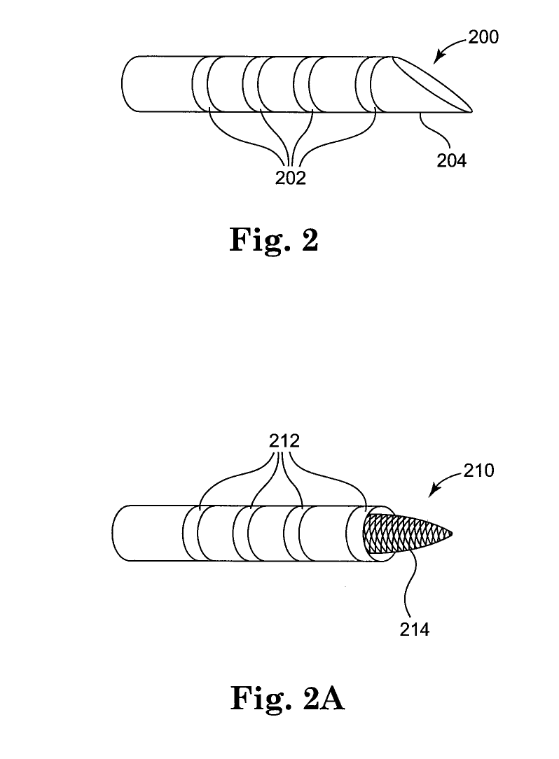

Referring now to FIG. 2, one embodiment of a septal puncture device 200 according to the current invention is illustrated. The device 200 is essentially the distal end of a puncture catheter or sharpened hypotube that can be delivered to the septum through the central lumens of the catheters of the current system. The device 200 has a plurality of evenly spaced radiopaque markers 202 on the distal portion and a relatively sharp puncture tip 204. The markers 210 can be observed under fluoroscopy during movement of the device, so that a clinician can make sure the device is properly oriented. Similarly, FIG. 2A is another embodiment of a septal puncture device 210, which includes multiple evenly spaced radiopaque markers 212 and an extendible obturator or puncture mechanism 214. The spacing on the markers can be used to guide the clinician while puncturing the septum, in that the distance that a given marker moves during the puncture procedure can be used as a guide to determine whether the septum has been punctured.

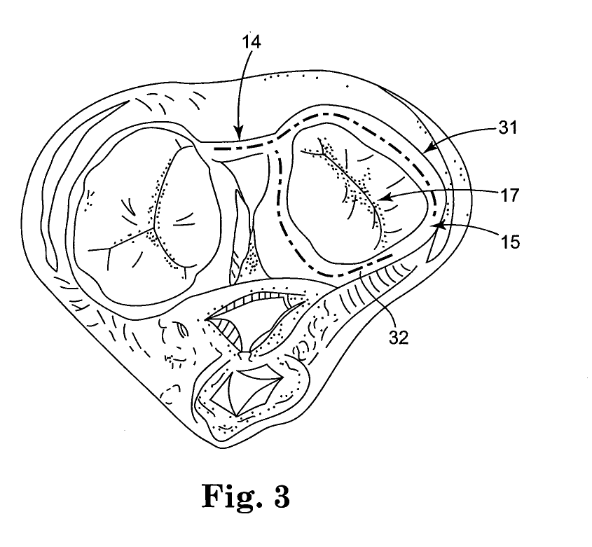

FIG. 3 illustrates one path that can be taken in mapping the shape and location of a mitral valve in the heart of a patient using the catheter systems of the invention. In this exemplary use of a catheter system, such as system 100, the septum is punctured through the interatrial septum 14, then the catheter system is placed in the left atrium adjacent the mitral valve 17 and used to map out the location of the annulus 15. In one embodiment, the catheter system encircles the entire annulus in a single procedure. In another embodiment, the catheter system is inserted around a first portion of the annulus, and then this or another device can be inserted around a second portion of the annulus. This method is illustrated by the dotted lines 31 and 32, which show the approximate orientation of a catheter during such a procedure.

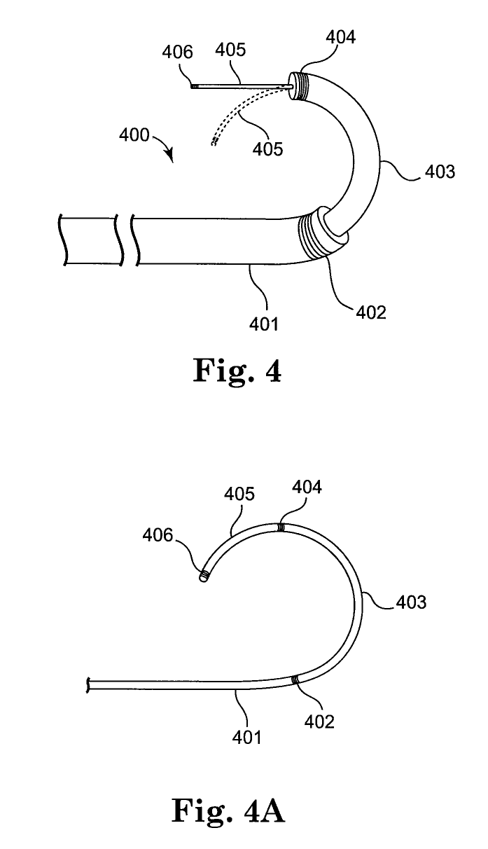

Referring now to FIG. 4, one embodiment of a catheter system 400 according to the invention is illustrated. The system 400 generally comprises a first flexible, articulating catheter 401, a second articulating catheter 403, and a wire 405. The catheters 401, 403 and wire 405 are coaxially positioned relative to each other, with the wire 405 being positioned within a lumen of catheter 403, which in turn is positioned within a lumen of catheter 401.

The first catheter 401 is made from one or more biocompatible polymeric materials that are appropriate for use within a human body. The body of the catheter 401 is sufficiently flexible to allow it to navigate the vasculature from an entry site to a location within the heart, such as the area of the mitral valve annulus. A first EM coil or other detectable marker 402 is located at the distal end of the catheter 401. When the marker 402 is an EM coil, the coil can be made from a thin wire made of a biocompatible metal, and the coil can have an inductance of over 70 microHenrys (pH). All of the coils of the currently described embodiments can be made from such materials and wrapped around the catheters and/or wires a sufficient number of times to have the necessary inductance. In one embodiment, the wire is wrapped around the catheters and/or guide wires 25 times. A thin communication wire (not shown) can be embedded in the catheter wall or affixed to the outside of the catheter 401, to conduct a charge between the coil 402 and an external AC power source (not shown). Suitable metals for the EM coil and the communication wire include, but are not limited to, copper, silver, gold, platinum and alloys thereof. In one preferred embodiment, the EM coil and the communication wire are both made from copper wires having a diameter of 0.001 inch (0.025 mm). In some cases, the communication may be done wirelessly, thereby eliminating the need for a communication wire.

The second flexible, articulating catheter 403 is disposed in the central lumen of the first catheter 401. The second catheter 403 is also made from one or more biocompatible materials appropriate for use in the human body. In this illustrated deployment configuration, a portion of the second catheter 403 extends beyond the distal tip of the first catheter 401. A second EM coil or other detectable marker 404 is located at the distal end of the second catheter 403. That is, when the marker 404 is an EM coil, the coil can be made from a thin wire made of some biocompatible metal, and the coil can have an inductance of over 70 pH. A thin communication wire (not shown) can be embedded in the catheter wall or affixed to the outside of the catheter 403 to conduct a charge between the coil 402 and the external AC power source connected to the first coil, or to a separate power source. Suitable metals for the second EM coil 404 and the communication wire include, but are not limited to, copper, silver, gold, platinum and alloys thereof. In one preferred embodiment, the EM coil and the communication wire are both made from copper wires having a diameter of 0.001 inch (0.025 mm).

The wire 405 is disposed in the central lumen of the second catheter 403. The wire 405 may be referred to as a guidewire, which can be a solid wire as generally described herein, although it also may include a central lumen or opening extending along at least a part of its length. In any case, the wire 405 is made from a biocompatible metal or alloy having suitable flexibility to navigate through the vascular system. Suitable metals/alloys include, but are not limited to, stainless steel, nitinol, MP35N, and others known to those skilled in the art. A third detectable marker or EM coil 406 is located on or at the distal tip of the wire 405, which can be the same or a different type of marker as the first and second markers 402, 404. The coil can be made from a thin wire made of some biocompatible metal and can have an inductance of over 70 μH.

In some embodiments of the invention, the wire (e.g., wire 405) is used as a guide wire, and it may also be used to transmit the charge to the EM coil (e.g., EM coil 406). In this case, the wire must also have suitable conductivity to transmit the electrical charge to the coil. In other embodiments, the wire is not used as a guide wire and is only used to transmit electricity to the coil and for mapping purposes. In one embodiment, a separate thin communication wire is affixed to the length of a guide wire and the communication wire transmits electricity to the coil. The wire and the EM coil can be made from the same type of metal, in one embodiment of the invention; however, the wire and the EM coil can instead be made from different types of metal.

Referring again to FIG. 4, the properties of the materials from which the catheters and wires are made (e.g., flexibility, shape properties, etc.) can be programmed into a processor so that an estimated shape can be computed prior to doing any actual mapping. When mapping the shape of a mitral valve, for example, a catheter system of the invention can be navigated to the left atrium, as described above, and the catheters 401 and 403 can be manipulated to conform to the shape of the valve annulus for mapping the shape of the mitral valve.

To begin the mapping process for a mitral valve, the catheter system 400 is first oriented so that its curved shape rests on either the posterior or anterior side of the mitral valve. Because the wire 405 is flexible, it can be advanced from the end of the second catheter 403 such that it will give a fuller depiction of the valve shape (as depicted by the position of the end of wire 405 shown as a dotted line). The shape of the entire annulus can be mapped without moving the system from the annulus, or the system can be rotated so that the curve rests on the other side of the valve.

FIG. 4A illustrates an exemplary view of how the curved catheter system of FIG. 4 can appear on a display device for an EM navigation system. The display device can be programmed to just display the location of the EM coils 402, 404, and 406 or it can show the entire system. The shape of the system can be determined by the processor based on the material characteristics of the catheters and wires, along with the recorded route traveled by the most distal coil. To further assist the clinician in precisely identifying each of the various markers of a single catheter system, the processor can be programmed to identify each of the catheters and/or wire in some manner that provides a unique identifier for each, such as different colors, brightnesses, and the like.

In one exemplary embodiment, the processor can be programmed to designate certain points along the valve annulus or other anatomical structure as reference points. The reference points can then be used as targets for implanting a therapeutic device, thus making sure that the device was precisely placed for optimal results. An example of such a procedure would be identifying and designating several points from one commissure to another, which in turn will help identify the valve leaflets. Once the location of the leaflets is identified, a properly shaped and sized therapeutic device (and tools for delivering it) can be selected.

In another exemplary use of the devices and methods of the invention, many points on the valve annulus and/or other structures are mapped to provide an accurate configuration of the annulus to the person selecting the optimally sized and shaped therapeutic device, such as a helical anchor device. An anchor device of this type can then be implanted on the valve annulus and adjusted to provide a desired size and shape for the annulus.

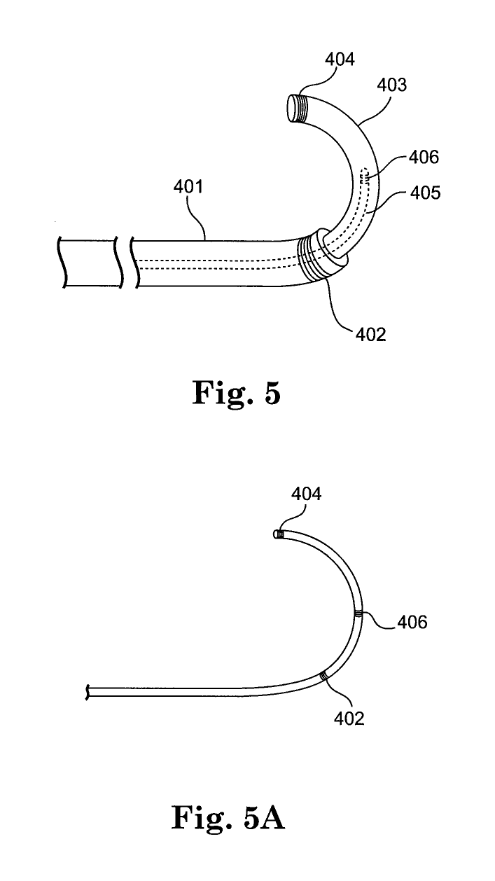

FIGS. 5 and 5A illustrate the catheter system of FIGS. 4 and 4A, respectively, with the wire 405 partially withdrawn into the central lumen of the second catheter 403. However, the wire 405 is only withdrawn, in this embodiment, until its EM coil 406 is positioned between EM coils 402 and 404 (i.e., its tip 406 is positioned in the area where the catheter 403 extends beyond the first catheter 401). FIG. 5A shows that when viewed on the display device, the EM coil 406 for the wire 405 can be seen between the EM coils 402 and 404 of the first catheter 401 and the second catheter 403, respectively. This feature of the catheter system takes advantage of the EM navigation characteristics to allow a clinician to gain a clearer understanding of the image on the display device. For example, once the general shape of the mitral vale annulus and the general location/orientation of the leaflets are determined, the clinician can move the wire 405 and use the position of its coil 406 to more accurately determine the location of specific valve structure and to designate reference points for implanting therapeutic devices.

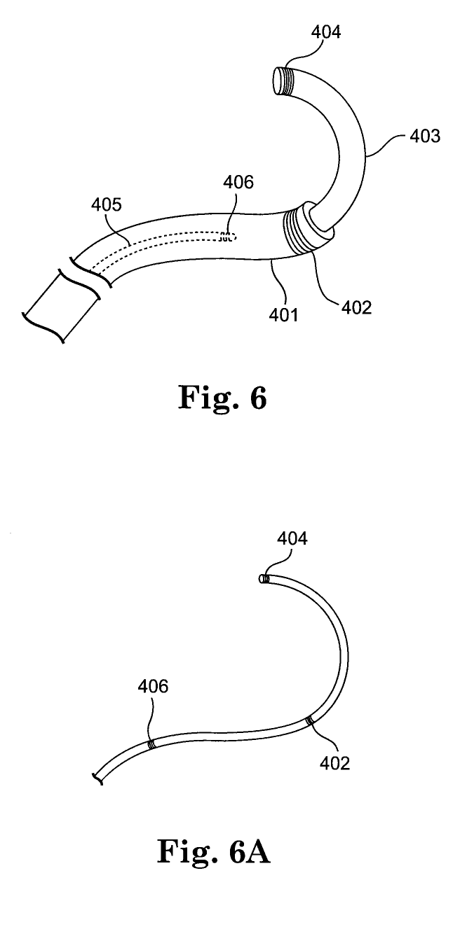

FIGS. 6 and 6A show another configuration of a catheter system of the current invention, with another positioning of the catheter system elements of FIGS. 4 and 4A, respectively. In this configuration, the wire 405 is withdrawn even further into the lumen of the second catheter 403 such that the distal most tip of the wire (represented by EM coil 406) is proximal of the distal tip (represented by EM coil 402) of the first catheter 401. Movement of the EM coil 406 (as illustrated in FIG. 6A) at the distal end of the wire 405, can be tracked in real time by tracking the position of the EM coil 406 relative to EM coil 402 to give a precise representation of the curvature of the catheter system. This feature allows a clinician to get an extremely accurate picture of the shape of a valve annulus or other structure by sliding the wire 405 back and forth relative to the lumen of the catheter.

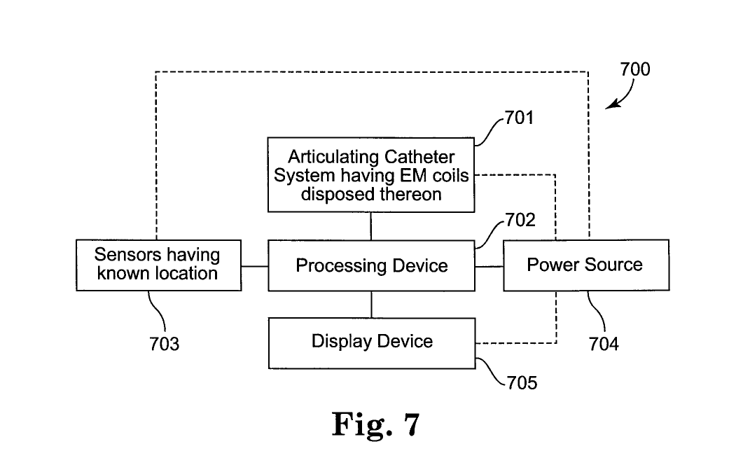

Referring now to FIG. 7, a system 700 is shown for mapping a valve annulus or other structure within a vascular system. In particular, the system 700 comprises a flexible articulating catheter system 701 having at least two flexible articulating catheters and a guidewire as previously described above. The articulating catheter system 701 can be attached to a processing device 702, which is also in signal communication with a plurality of sensors having a known location 703 and a display device 705. A power source 704 provides power to the processing device 702, and it can also provide power to each of the other components of the system through the processing device 702 or separately. In alternate embodiments of the system, each component can have its own separate power source. In one embodiment, the catheter system 701 is not connected to the processing device.

One method of using the current invention involves placing a telescoping catheter having EM coils into the vascular system of a patient. The catheter can be navigated through the vascular system and into the left atrium of the patient. The distal end of the catheter is manipulated to mimic the shape of the mitral valve. During the procedure, the EM coils emit signals that are detected by a plurality of sensors arranged under and/or around the patient. The sensors transmit information about the signal strength and direction to a processor that then calculates the location of each EM coil relative to each sensor, which can be determined in real time. The processor then displays the information on a display device in a form useful to the clinician performing the procedure.

The current invention includes within its scope systems for determining the shape of a mitral valve or other vascular structure using EM navigation techniques. While the devices in this disclosure have been discussed in terms of having transmitters on a catheter system and receivers/sensors outside of a patent's body, this can be reversed such that the sensors are on the catheter system and the transmitters are outside the body. Alternatively, a system can be provided in accordance with the invention where both transmitters and sensors are on the catheter system, and both transmitters and sensors are located outside of the patent's body.

The currently disclosed devices can also be connected to a DC power source and used in an electro-potential (EP) navigation system in a similar manner to that described above. The devices and methods disclosed herein can also be used in combination with other visualization/imaging devices and methods to provide a clinician with a detailed understanding of a particular patient's vasculature.

Some embodiments of the devices disclosed herein can include materials having a high X-ray attenuation coefficient (radiopaque materials). The devices may be made in whole or in part from the material, or they may be coated in whole or in part by radiopaque materials. Alloys or plastics may include radiopaque components that are integral to the materials. Examples of suitable radiopaque material include, but are not limited to gold, tungsten, silver, iridium, platinum, barium sulfate and bismuth sub-carbonate.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. An image guided navigation system for mapping at least one of the shape, size and location of a structure within a heart of a patient, the system comprising:
a catheter system comprising a first catheter comprising a longitudinally extending lumen, a second catheter comprising a longitudinally extending lumen and positioned at least partially within and moveable relative to the lumen of the first catheter, an elongated member positioned at least partially within and moveable relative to a lumen of the second catheter, a first detectable marker located at a generally distal end of the first catheter, a second detectable marker located at a generally distal end of the second catheter, and a third detectable marker located at a generally distal end of the elongated member;
wherein at least one of the three detectable markers is axially moveable relative at least one of the other detectable markers.

2. The system of claim 1, wherein at least one of the first, second, and third detectable markers comprises an electromagnetic coil.

3. The system of claim 2, wherein all three of the detectable markers are electromagnetic coils.

4. The system of claim 1, wherein the detectable marker of the elongated member is extendable past the distal end of the second catheter.

5. The system of claim 1, wherein the detectable marker of the second catheter is extendable past the distal end of the first catheter.

6. The system of claim 1, further comprising a processor for calculating a position of each of the first, second, and third detectable markers.

7. The system of claim 1, further comprising a puncturing mechanism for accessing a predetermined area of a patient.

8. The system of claim 1, further comprising a display for receiving location data for each of the three detectable markers and displaying the location data in a visible manner on the display.

9. The system of claim 1, wherein the elongated member is a guidewire.

10. The system of claim 1, wherein the elongated member is a third catheter comprising a longitudinally extending lumen.

11. A method of determining and mapping at least one of a shape and a location of a predetermined anatomical structure, the method comprising the steps of:
providing a telescoping catheter system comprising at least two coaxially positioned catheters and an elongated member coaxially positioned within the two coaxially positioned catheters, wherein a distal end of each of the catheters and the elongated member comprises a detectable marker;
inserting the catheter system into a patient to the location of the predetermined anatomical structure;
axially moving at least one of the detectable markers relative to at least one other of the detectable markers; and
detecting the relative movement of the markers with at least one sensor.

12. The method of claim 11, further comprising the steps of transmitting information received from the at least one sensor to a processor, and calculating the location of each detectable marker relative to the at least one sensor.

13. The method of claim 12, further comprising the step of displaying the location of each detectable marker on a display device.

14. The method of claim 11, further comprising the step of moving only the detectable marker of the elongated member relative to the detectable markers of the catheters.

15. The method of claim 11, wherein at least one of the detectable markers comprises an electromagnetic coil.

16. The method of claim 11, wherein the predetermined anatomical structure is a valve of a beating heart.

17. The method of claim 16, wherein the valve is a mitral valve.

18. The method of claim 12, wherein each of the detectable markers comprises a unique identifier so that the processor can individually identify each of the detectable markers.

19. The method of claim 11, wherein the elongated member is a guidewire.

20. The method of claim 11, wherein the elongated member is a third catheter comprising a longitudinally extending lumen.
